(19) United States
(12) Patent Application Publication
(10) Pub. No.: US 2008/0015140 A1
(43) Pub. Date: Jan. 17, 2008

(54) DISINFECTING COMPOSITION

(75) Inventors: Vladimir Semenovich Kuzmin, Moscow (RU); Oleg Ivanovich Lukashov, Moscow (RU); Victor Stanislavovich Polyakov, Moscow (RU); Valeriy Vasilievich Ermilov, Moscow (RU)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Veckis Industries Ltd., Vaduz (LI)

(21) Appl. No.: 11/766,264

(22) Filed: Jun. 21, 2007

Related U.S. Application Data
(63) Continuation of application No. 11/004,768, filed on Dec. 3, 2004, now abandoned.
Application No. 11/004,768 is a continuation of application No. 10/185,024, filed on Jun. 28, 2002, now abandoned.

(51) Int. Cl. A61K 38/16; A61K 31/28

(52) U.S. Cl. 514/6; 514/500

(57) ABSTRACT

The invention relates to preparations that can be used for disinfecting and that can be applied in the national economy, medicine, and laboratories of all types. The preparation contains a chelating metal complex compound with a monodentate, bidentate, or polydentate ligand that exhibits affinity to hydrogen ion, an ionogenic surfactant, and a solvent. The preparation displays antiseptic properties. The preparation inhibits Gram-positive and Gram-negative bacteria, viruses, and spores. The preparation can be applied in a broad temperature range.

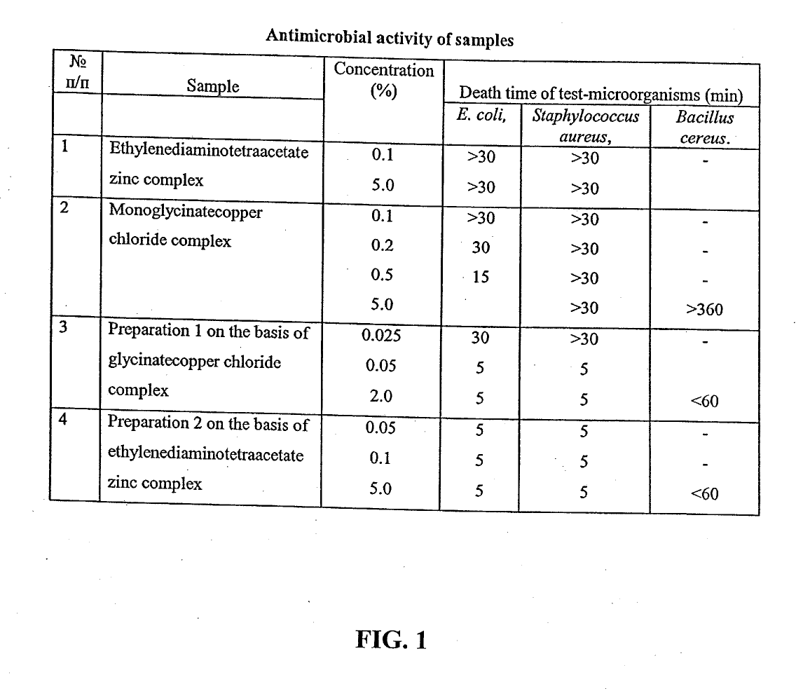

### CROSS-REFERENCE TO RELATED APPLICATIONS

This application is a continuation application of U.S. application Ser. No. 11/004,768, filed Dec. 3, 2004, which is a continuation application of U.S. application Ser. No. 10/185,024, filed Jun. 28, 2002, both of which are incorporated herein by reference in their entirety.

### FIELD OF THE INVENTION

The invention relates to compositions useful for disinfecting and intended for use in food industry and different sectors of national and municipal economy, public and utility services, public catering establishments, agriculture, medicine, laboratories of all types, private life et al. The proposed compositions can be applied as a universal disinfecting, sterilizing and bactericidal agents in different areas of national economy.

### BACKGROUND OF THE INVENTION

One of the well known disinfecting agents is hydrogen peroxide and preparations thereof. A representative of this group is a disinfecting preparation containing hydrogen peroxide, magnesium laurylsulphate, glycerin, sodium oleate, disodium salt of EDTA, sodium benzoate and water (RU2108810 C1, 1998). This agent is intended for decontaminating surfaces of houses, sanitary appliances, linen, medical goods and its efficacy is not sufficient. It is not toxic for men and animals.

Broadly known are bactericidal compositions containing lanthionine and a chelating agent, exhibiting an increased activity. The suitable chelating agents are for example ethylenediaminotetraacetic acid (EDTA), its salts and citrate. (U.S. Pat. No. 5,260,271, 1993; U.S. Pat. No. 5,334,582, 1994).

Also known are a bactericide, which represents a composition, including a metal complex with an α-amino acid and obtained in an acidic medium, and a disinfectant. (U.S. Pat. No. 6,242,009, 1999).

It is known that chelating metal complexes exist in an acidic medium only in negligible concentrations. (Fundamentals of Analytical Chemistra Book 1, Moscow—“Mir”—D. Skoog, D. West, 1979).

For example, a chelating agent as EDTA completely binds metal ions to form chelating complexes at pH above 6.0. For weaker chelating agents, to which natural amino acids are an example, to completely bind all metal ions into chelating complexes, the pH values of media should not be higher. The investigations carried out by the inventors have revealed that in the U.S. Pat. No. 6,242,009 transformation of amino acids and metal ions into nondissociating chelating complexes can occur only at pH>7.0 and addition of mineral acids in accordance with the examples cited in the patent leads to the destruction of the chelating complexes. In addition, the amino group of the amino acid is protonated and the metal exists in an ionic form. Antimicrobial activity of the compounds cited in U.S. Pat. No. 6,242,009 can be attributed not to the activity of chelating complexes but to metal ions, which, as is known from the literature, also exhibit certain bactericidal activity, in particular, the cited silver ions. It should be also noted that arsenic and selenium compounds are cited in the U.S. Pat. No. 6,242,009 as perspective ones and their antibacterial activity can be determined by a high toxicity to all living organisms, humans included. There is no doubt that the presence of strong disinfectants (chlorohexydine, hydrogen peroxide), which are introduced as additives to the complexes cited in U.S. Pat. No. 6,242,009, can increase the activity of the preparation.

Also described are bactericide compositions, which include cetyltrimethylammonium chloride as an active compound (DE 4326866, 1995; U.S. Pat. No. 5,206,016, 1993; U.S. Pat. No. 5,575,991, 1996).

Of interest is an antiseptic preparation, which includes as an active compound cetyltrimethylammonium chloride, a mineral or an organic acid and a solvent (RU 2118174 C1, 1998). The known compound exhibits bactericidal activity towards gram negative microflora and it is not substantially effective towards intestinal and other infections of bacterial and viral etiology as well as towards anthrax.

Also known is a disinfecting preparation containing bacteriocine, a chelating agent, a stabilizer, a surfactant, a salt and an alcohol (RU 2163145, 1999). The known preparation is used for impregnating napkins, which are applied for prophylaxis of mastitis in animals.

The most closely related compound to the present one is a disinfecting preparation, which contains an active compound—a peroxide compound, a surfactant, a chelating complex and a solvent (RU20614497, 1996). This compound is active only when used at positive temperatures of 18-25° C. The prolongation of the bacteria inactivation is varied in the interval of 5-30 minutes.

### SUMMARY OF THE INVENTION

The invention is based on the objective of creating a universal highly effective disinfecting, antiseptic and bactericidal preparation, useful in a broad range of positive and negative temperatures, and in increasing the term of bactericidal action, which is suitable for a long-term storage, which is safely used, which exhibits high bactericidal, virucidal, fungicidal, and sporocidal activity and which is nontoxic.

The present invention comprises a disinfecting preparation including an ionogenic surfactant, a chelating complex and a solvent. According to the invention, the chelating complex comprises a metal compound, containing a monodentate bidentate or polydentate ligand, which exhibits affinity towards hydrogen ion, and together with the surfactant is in the proportion of 1-(7-9) to the solvent. An exemplary comprises solvent of distilled water.

The chelating metal complex compound containing the ligand of this invention is a chelating complex compound with a metal selected from copper, zinc, mercury, chromium, manganese, nickel, cadmium, arsenic, cobalt, aluminum, lead, selenium, platinum, gold, titanium, tin or combinations thereof.

The bi- and polydentate ligands are, for example, selected from anions of natural amino acids, iminodiacetic or nitriletriacetitic acids as well as carbon-substituted (in the χ-position to the carboxylic group) derivatives of iminodiacetic and nitriletriacetic acids with various remnants of amino acid fragments containing no aminocarboxylic group, alkylenediaminopolyacetic acid, as well as carbon-substituted (in the χ-position to the carboxylic group) derivatives of polyalkylenepolyaminopolyacetitc acids with various remnants of aminoacetic fragments containing no aminocarboxylic group, derivatives of ω-phosphoncarboxylic and ethylenediphosphontetrapropionic acids, derivatives of ethelynetetra(thioacetic) and diethylenetrithiodiacetic acids, monoamine complexones, in which carboxylic groups are replaced by phosphonic groups, or mixtures thereof.

The chelating metal complex compound containing a monodentate ligand can be a chelating complex compound with at least one amino acid such as, for example, isoleucine, phenylalanine, leucine, lysine, methionine, threonine, tryptophan, valine, alanine, glycine, arginine, histidine, or mixtures thereof.

One embodiment of the invention comprises a disinfectant composition containing an ionogenic surfactant, a chelating complex and a solvent, wherein the chelating complex comprises a chelating metal complex compound containing a monodentate, bidentate or polydentate, ligand, which exhibits affinity to hydrogen ion, and the solvent comprises a mixture of water and an aliphatic alcohol (C₁-C₈ ) with the following ratio, weight %:

| | |
|---|---|
| Chelating complex metal compound, | 1-30 |
| containing a monodentate ligand which | |
| exhibits affinity to hydrogen ion | |
| Ionogenic surfactant | 0.1-15 |
| Aliphatic alcohol (C₁-C₈) | 0.5-95 |
| Distilled water | the rest |

Another aspect of the invention comprises a disinfecting composition comprising an ionic surfactant, a chelating complex and a solvent. The chelating complex comprises a chelating complex metal compound, which includes along with commonly used bi- and polydentate ligand an additional monodentate ligand exhibiting affinity towards hydrogen ion, and exemplary solvents, include distilled water and an aliphatic alcohol (C₁-C₈) with the following weight % ratio:

| | |
|---|---|
| Chelating metal complex compound, | 1-30 |
| containing a monodentate ligand | |
| and exhibiting affinity towards hydrogen ion- | |
| Ionogenic surfactant- | 0.1-15 |
| Aliphatic alcohol (C₁-C₈)- | 0.5-95 |
| Distilled water- | remainder |

An exemplary chelating metal complex compound comprises glycinatecopper chloride complex.

A further exemplary chelating metal complex compound, is the ethylenediaminotetraacetate zinc complex.

Exemplary ionogenic surfactants comprises cetylpyridinium halogenides. Additional such surfactants are the cetyltrimethylammonium halogenides.

Metal complex compounds are useful disinfecting and antibacterial preparations. They are bactericidal reagents exhibiting a broad range of antibacterial action, irreversibly killing a pathogenic microflora. The mechanism of action of metal complex compounds is based on blocking amino acid groups of a protein shell and enzyme systems of microorganisms. At the first stage there are formed associates with a chelating complex and then a monodentate ligand is substituted by an amino acid group of protein, which leads to a complete blocking of metabolic processes in microorganisms and to their death.

By the toxic action on a human organism the proposed compounds relate to the IY class of danger. Doses, which are used in practice for antibacterial treatment, do not cause a pronounced toxic or irritating effect on skin or mucosa.

The proposed compositions based on chelating metal complex compounds do not exert influence on a human organism because the compounds containing amino acid groupings are withdrawn from the organism by the exchange reaction. Bactericidal chelating complexes practically do not affect the most important living functions of the organism.

The proposed bactericides relate to metal complexes with chelating ligands, which are obtained in the alkaline and not in the acidic pH range. Therefore, the proposed compositions compared to the analogs have a broader field of application because they are ecologically safe and possess low toxic and hygienic characteristics based on a different mechanism of bactericide action. In addition the proposed compositions exhibit an increased chemical stability towards environmental impact (stability constants of the proposed complexes are several orders higher than those of the closest analogs).

Useful monodentate ligands include ligands exhibiting affinity towards hydrogen ion, which determines their ability to be substituted by an amino group of protein in a microorganism.

A molecule of the proposed bactericide contains a metal ion preferably, for example, copper (II) and zinc as well as monodentate ligands, exhibiting affinity towards hydrogen ion, such as ammonia, mono-, di- and triethanolamines and others.

The pH of the obtained bactericidal compositions is ≧7.0.

For the synthesis of bactericides, use is made of metal salts. The synthesis is carried out in aqueous solutions by stirring the ingredients at room temperature. The monodentate ligands used are water soluble substances which display affinity towards a hydrogen ion.

The distinguishing characteristic of the present bactericide compositions is that the interaction (mixing) of the ingredients takes place in neutral and alkali media at pH≧7.0 in the absence of mineral acids.

As for the parameters of the disinfecting activity, it is established that the present bactericidal compositions are sufficient and do not require the use of any additional disinfecting preparations, for example, chlorohexydine, hydrogen peroxide et al.

The method for synthesis of the glycinatecopper chloride complex and ethylenediaminotetraacetate zinc complex is known from the following sources:
Ley, Berichte, V. 42, S. 371;
Hofmeister, “Beittage zur Kenntiniss der Amidosäurcn” Annalen der Chemie, 1877 V. 189, S. 36;
“Synthetic Production and Utilization of Amino Acids,” Ed. T. Kaneko, Y. Izumi, I. Chibata, Wiley, N.-Y., 1974.
Dyatlova N. M. et al., Complexones and Metal Complexonates, M.:-<<Khimiya>> 1988.

Antimicrobial activity of the glycinatecopper chloride complex, ethylenediaminptetraacetate zinc complex and compositions thereof was investigated in the Scientific Research Disinfectology Institute, Moscow (the data are given in the report of the Institute of 15.02.2002).

The ingredients ratio in the proposed compositions is selected such that to provide for optimal technological characteristics of the preparation and for retaining the stable properties.

The concentrations ranges in the compositions:

| | |
|---|---|
| Chelating metal complex | 1%-30% |
| Ionogenic Surfactant (quaternary ammonium halogenides-, | 0.1%-15% |
| C₁₂-C₁₆-alkyltrimethylammonium, di(C₈-C₁₀ | |
| -alkyl)dimethylammonium, in particular cetylpyridinium and | |
| cetyltrimethylammonium halogenides | |
| Aliphatic alcohol (C₁-C₈) | 0.5%-95% |
| Water | 3%-98% |

The proposed concentration ranges for the ingredients in the composition are determined by the object to achieve the above mentioned bactericidal, fungicidal and sporocidal efficiency of the composition.

The technical result is possible to achieve by making use of—as ionogenic surfactants—quaternary ammonium halogenides, in particular C₁₂-C₁₆ alkyltrimethylammonium, di(C₈-C₁₀-alkyl)dimethylammonium, C₁₂-C₁₆-alkylpyridinium, in particular cetylpyridinium and cetyltrimethylammonium halogenides.

Industrial application of the proposed preparation is confirmed by the following examples.

### EXAMPLE 1

2.0 g of sodium hydroxide is dissolved in 50 cm³ of distilled water in a flask and 3.75 g of glycine is added on stirring. 6.8 g of zinc chloride is added portion-wise to the obtained solution on stirring followed by the addition of 3.75 cm³ of 25% aqueous solution of ammonium. Separately there is prepared a solution of 0.43 g of cetyltrimethylammonium chloride in the mixture of 1.2 g of tryethyleneglycol and 15.3 cm³ of water. Both solutions are mixed and diluted with water to achieve the concentration which is required for the antibacterial treatment of objects.

### EXAMPLE 2

To 6.1 cm³ of 25% solution of ammonia in a flask there are added 25 ml of water and 11.85 g of ethylenediaminotetraacetic acid. On stirring, there is added portion-wise 5.45 g of copper dichloride and 2.4 g of ethanolamine is poured. The formed solution turns dark blue. Separately there is prepared a solution of 8.1 g dodecylbenzyltrimethylammonium chloride in a mixture of 7.3 cm³ of isopropyl alcohol and 10 cm³ of water. Both solutions are mixed and diluted to achieve the concentration required for the antibacterial treatment of objects.

### EXAMPLE 3

In a flask 0.4 g of sodium hydroxide is dissolved in 20 cm³ of distilled water and 1.46 g of L-lysine is added on stirring. Then 1.36 g of zinc chloride is added portion-wise on stirring. The obtained solution is mixed with 0.75 cm³ of 25% solution of ammonium in water. Separately there is prepared a solution of 12.0 g of cetylpyridinium chloride in 56.0 cm³ of isopropyl alcohol. An aqueous solution of a zinc amino acid complex is added slowly, portion-wise. The mixture is stirred and diluted with water to achieve the concentration, which is required for the antibacterial treatment of objects.

### EXAMPLE 4

A chelating metal complex compound containing a monodentate ligand, which displays affinity towards hydrogen ion, is mixed with an ionogenic surfactant, in particular as is indicated in Example 1. Distilled water is added to achieve the 10% or 30% concentration, i.e. the ratio with the solvent of 1-9 or 7.

### EXAMPLE 5

The ingredients are mixed as is described in Example 2 in the following amounts (%):

| | |
|---|---|
| Chelating metal complex compound containing a | −30 |
| monodentate ligand, which displays affinity towards | |
| hydrogen ion | |
| Ionogenic surfactant | −15 |
| Aliphatic alcohol (C₁-C₈) | −0.5 |
| Distilled water | −54.5 |

### EXAMPLE 6

The ingredients are mixed as is described in Example 2, in weight %:

| | |
|---|---|
| Chelating metal complex compound containing a | −2 |
| monodentate ligand, which displays affinity towards | |
| hydrogen ion | |
| Ionic surfactant | −1 |
| Aliphatic alcohol (C₁-C₈) | −95 |
| Distilled water | −2 |

### EXAMPLE 7

The ingredients are mixed as is described in Example 3 in the following mass %:

| | |
|---|---|
| Chelating metal complex compound containing a | −1 |
| monodentate ligand, which displays affinity towards | |
| hydrogen ion | |
| Ionogenic surfactant | −5 |
| Aliphatic alcohol (C₁-C₈) | −20 |
| Distilled water | −74 |

### EXAMPLE 8

The ingredients are mixed as is described in Example 3 in the following mass %:

| | |
|---|---|
| Chelating metal complex compound containing a | −2 |
| monodentate ligand which displays affinity towards | |
| hydrogen ion | |
| Ionogenic surfactant | −0.1 |
| Aliphatic alcohol (C₁-C₈) | −30 |
| Distilled water | −67.9 |

### Bactericidal Activity

To investigate the disinfecting properties of samples, as test microorganisms, use was made of vegetative forms of bacteria E. coli (strain 1257), which simulates pathogens of intestinal infections—gram negative bacteria; Staphylococcus aureus (strain 906), which simulates infections of respiratory tract and is a pathogen of hospital infections—gram positive bacteria, as well as of bacteria Bacillus cereus (strain 96), which simulates an anaerobic infection—gas gangrene, tetanus and anthrax.

The initial investigations of chelating metal complexes, for example of glycinatecopper ammonium chloride, have revealed their high enough efficacy towards the vegetative forms (see Table 1).

For increasing antibacterial efficacy, in particular sporocidal properties, ionogenic surfactants (cetylpyridinium chloride, cetyltrimethylammonium bromide) were introduced into the solutions of chelating metal complex compounds. Thus the obtained composition on the basis of glycinatecopper ammonium chloride and cetyltrimethylammonium bromide (preparation 1, see Table 1) displays the synergism of action towards gram negative and gram positive bacteria.

Among the preparations on the basis of chelating zinc complexes, the highest activity towards the aforementioned types of bacteria is displayed by preparation 2 (see Table 1), which is based on 2-aminoethanol ethylenediaminotetraacetate zinc complex and cetylpyridinium chloride.

TABLE 1
Antimicrobial activity of samples

| | | | Death time of | | |
|---|---|---|---|---|---|
| | | | test-microorganisms (min) | | |
| Na | | Concentration | | Staphylococcus | Bacillus |
| n/u | Sample | (%) | E. coli. | aureus. | coreus. |
| 1 | Ethylenediaminotetraacetate | 0.1 | >30 | >30 | — |
| | Zinc complex | 5.0 | >30 | >30 | |
| 2 | Monoglycinatecopper | 0.1 | >30 | >30 | — |
| | chloride complex | 0.2 | 30 | >30 | — |
| | | 0.5 | 15 | >30 | — |
| | | 5.0 | | >30 | >360 |
| 3 | Preparation 1 on the basis of | 0.025 | 30 | >30 | — |
| | glycinatecopper chloride | 0.05 | 5 | 5 | |
| | complex | 2.0 | 5 | 5 | <60 |
| 4 | Preparation 2 on the basis of | 0.05 | 5 | 5 | — |
| | ethylenediaminotetraacetate | 0.1 | 5 | 5 | — |
| | Zinc complex | 5.0 | 5 | 5 | <60 |

The investigations of the preparation, which consists of 5% solution of ethylenediaminotetraacetate zinc in a water-alcohol solution (70 vol. % isopropyl alcohol), have revealed activity towards vegetative types of bacteria on a 128-fold dilution, while towards anthrax (spores)—on a 16-fold dilution.

The proposed universal ecologically safe bactericidal preparation is intended for disinfecting the main forms and types of pathogenic microflora including the spore form. The preparation exhibits increased ecological properties, which is achieved by applying nontoxic chelating agents transforming metal ions into nontoxic chelating complexes.

The preparation makes it possible:
To reduce the cost of the bactericide complex;
To increase environmental stability due to the fact that the proposed bactericide chelating metal complexes are independent on such environmental factors as temperature, humidity, light effect;
To retain operating properties for many years.

In the present investigations, there is established that bactericidal effect and stability of the preparation are decreased in case the ingredients content is lower than the pointed minimal values of the composition.

Stability of the preparation is decreased on using concentrations of the ingredients above the maximum values.

The present standard investigations have revealed high efficiency of the preparation towards such pathogens as:
Intestinal infections (gram negative bacteria)—pseudomonas aeruginosa, dysenteria, salmonellosis;
Respiratory tract and hospital infections (gram positive bacteria)—staphylococcosis, streptococcosis, microflora et al.;
Anaerobic infections—wound infections (tetanus);
Anthrax (spores) et al.

The preparation effectively acts on viruses (hepatitis, herpes, AIDS-infection, rotaviral infections).

Buffering of the bactericide composition provides for the desirable bactericidal effect at all pH values of a human skin, the pH value of the preparation is weakly alkaline (i.e., pH 7.6±0.5).

The area of application of the preparation is that of prophylaxis and disinfecting of contaminated open parts of human and animal skin as well as of surfaces of the majority of materials.

By its content and principal of action, the preparation is safe for humans and animals, nontoxic, does not irritate skin, chemically neutral towards all construction materials and fabrics based on natural and synthetic fibers, does not cause corrosion of metals.

If the preparation is applied over skin, the bactericide effect is retained for not less than 2 hours; while applied over surfaces of materials, fabrics, and protective coverings—24 hours and above.

The temperature range for skin application is from −20° C. to +40° C.-+50° C.; for surfaces −50° C. to +50° C. The preparation kills 99.99% of microbes. By acute toxicity, the preparation is related to the IY class of low hazard compounds.

A mixture of effective amounts of ingredients exhibits a synergetic effect and disinfecting properties are increased.

That which is claimed:

1. A disinfectant composition comprising an ionogenic surfactant, a glycinate copper halide complex, and isopropyl alcohol, and wherein the ionogenic surfactant and the glycinate copper halide complex are present in disinfectant effective amounts.

2. A disinfectant composition comprising an ionogenic surfactant, a chelating complex and isopropyl alcohol, wherein the chelating complex comprises a chelating metal complex compound containing a bidentate or polydentate ligand that exhibits affinity to hydrogen ion, wherein the bidentate and polydentate ligands are selected from the group consisting of anions of natural amino acids, iminodiacetic or nitrilotriacetic acids as well as carbon-substituted (in the χ-position to the carboxylic group) derivatives of iminodiacetic and nitrilotriacetic acids with various remnants of amino acids fragments containing no aminocarboxylic group, alkylenediaminopolyacetic acid, as well as carbon-substituted (in the X-position to the carboxylic group) derivatives of polyalkylenepolyaminopolyacetitc acids with various remnants of aminoacetic fragments containing no aminocarboxylic group, derivatives of ω-phosphoncarboxylic and ethylenediphosphontetrapropionic acids, derivatives of ehtelynetetra(thioacetic) and diethylenetrithiodiacetic acids, monoamine complexones, in which carboxylic groups are replaced by phosphonic groups, and mixtures thereof, and wherein the ionogenic surfactant and chelating complex are present in disinfectant effective amounts.

3. A disinfectant composition comprising an ionogenic surfactant, a chelating complex and isopropyl alcohol, wherein the chelating complex comprises a chelating metal complex compound containing a monodentate ligand that exhibits affinity to hydrogen ion, wherein the chelating metal complex compound containing a monodentate ligand is a chelating complex compound with at least one amino acid selected from the group consisting of isoleucine, phenylalanine, leucine, lysine, methionine, threonine, tryptophan, valine, alanine, glycine, arginine, histidine, and mixtures thereof, and wherein the ionogenic surfactant and chelating complex are present in disinfectant effective amounts.

4. A disinfectant composition comprising an ionogenic surfactant, a glycinate copper halide complex, and isopropyl alcohol, with the following ratio, weight %:

| | |
|---|---|
| Glycinate copper halide complex | 1-30 |
| Ionogenic surfactant | 0.1-15 |
| Isopropyl alcohol | 0.5-95 |
| Distilled water | the rest |

wherein the ionogenic surfactant and glycinate copper halide complex are present in disinfectant effective amounts.
